SEQUENCE LISTING (2 sequences)

SEQ ID NO: 1 - DNA, 35 bp, Artificial Sequence
  Primer 24885
ccagctggtacacagattacttgcacgggaaatgc

SEQ ID NO: 2 - DNA, 22 bp, Artificial sequence
  Primer 130411jvi8
ggcatcattgttttttccgcag

(19) United States
(12) Patent Application Publication
(10) Pub. No.: US 2015/0307871 A1
(43) Pub. Date: Oct. 29, 2015

(54) METHOD FOR GENERATING SITE-SPECIFIC MUTATIONS IN FILAMENTOUS FUNGI

(71) Applicant: NOVOZYMES A/S, Bagsvaerd (DK)

(72) Inventor: Jesper Vind, Bagsvaerd (DK)

(21) Appl. No.: 14/648,486

(22) PCT Filed: Dec. 2, 2013
(86) PCT No.: PCT/EP2013/075227

§ 371 Date: May 29, 2015

(30) Foreign Application Priority Data
Dec. 7, 2012 (EP) 12195995.1
Apr. 9, 2013 (EP) 13162884.4

(51) Int. Cl. C12N 15/10

(52) U.S. Cl. CPC C12N 15/1024

(57) ABSTRACT

The present invention provides methods of making site-directed mutations in a gene encoding a polypeptide of interest to be transformed directly into a filamentous fungal host, without having to rely an intermediate host like E. coli to generate sufficient genetic material to successfully transform the fungal host.

### REFERENCE TO A SEQUENCE LISTING

This application contains a Sequence Listing in computer readable form, which is incorporated herein by reference.

### FIELD OF THE INVENTION

The present invention relates to methods for providing a site-specifically mutated variant polypeptide of interest.

### DESCRIPTION OF THE RELATED ART

Several methods of making site-directed mutations in polypeptides of interest are known, including, for example, splicing by overlap extension PCR (“SOE-PCR”) or the QuickChange® method (Stratagene Inc.). The latter employs a set of overlapping mutagenic PCR primers to amplify a methylated double-stranded plasmid template in a linear PCR reaction followed by self-ligation of the fragments, where Dpn1 nuclease is used to remove methylated template plasmid after conducting the PCR reaction and before transforming the ligation mixture into an E. coli host. However, there is a constant need to improve transformation and selection efficiency, especially in filamentous fungal hosts.

### SUMMARY OF THE INVENTION

The present invention provides methods of making site-directed mutations in a gene encoding a polypeptide of interest to be transformed directly into a filamentous fungal host, without having to rely an intermediate host like E. coli to generate sufficient genetic material to successfully transform the fungal host. These methods involve using a specifically methylated autosomal replicating plasmid comprising the encoding gene as template in a PCR reaction with a pair of non-overlapping end-to-end primers, wherein at least one primer is mutagenic, followed by removal of the methylated template DNA, self-ligation to re-circularize the PCR fragments and direct transformation of the resulting re-circularized vectors into the filamentous fungal expression host of choice, wherein the primers are either phosphorylated prior to the PCR reaction or the resulting PCR fragment is phosphorylated before or during the ligation step to enable successful ligation.

Accordingly, in a first aspect, the invention relates to a method of providing site-specifically mutated variant polypeptides, the method comprising the steps of:
a) providing a methylated template autosomal filamentous fungal replicating double-stranded circular DNA vector comprising a parent polynucleotide encoding a parent polypeptide;
b) providing a pair of end-to-end non-overlapping PCR primers directed to the parent polynucleotide, one 5′ forward primer and another 3′ reverse primer, wherein at least one primer is mutagenic;
c) performing a PCR amplification of the template vector with the pair of PCR primers to generate full-length vector mutated PCR fragments;
d) removing the template vector with a suitable methylation-specific nuclease;
e) circularizing the mutated PCR fragments by self-ligation; and
f) transforming the circularized mutated PCR fragments directly into a filamentous fungal host cell to express the variant polypeptides,
   wherein either the PCR primers are phosphorylated prior to the PCR amplification, or the PCR fragments are phosphorylated before or during the self-ligation step to allow end-to-end ligation of the primers to circularize the mutated PCR fragments.

### BRIEF DESCRIPTION OF THE FIGURES

FIG. 1 shows a schematic map of template plasmid pEN14286 used in Example 1.

FIG. 2 shows a picture of an agarose gel used to verify the size of the PCR fragments generated in Example 1.

FIG. 3 shows a picture of an SDS-page gel used in Example 1 to verify the successful removal of a glycosylation site by the claimed site-directed mutagenesis method. The details of the gel are discussed in the end of the Example.

### DEFINITIONS

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

End-to-end non-overlapping PCR primers: This term means a pair of PCR primers, one 5′ forward primer and one 3′ reverse primer which target a continuous polynucleotide region on a double-stranded circular DNA vector template, where the primers line up end-to-end when each primer is annealed to its template strand without any basepair overlap between them. In this manner the two primers will each represent the ends of a fully amplified doublestranded vector PCR fragment which may be ligated together to re-circularize the vector if either the primers are phosphorylated prior to the PCR amplification or the PCR fragment is phosphorylized before or during ligation. When at least one of the end-to-end non-overlapping PCR primers is mutagenic, i.e., it comprises at least one nucleotide that is different from the template polynucleotide region to which said mutagenic primer is targeted, then that mutation will be incorporated into the resulting amplified PCR fragment.

Methylated template autosomal filamentous fungal replicating double-stranded circular DNA vector: This term means a regular double-stranded circular DNA vector, i.e. a plasmid, which is methylated, autosomal and capable of independent replication in a filamentous fungal host cell, for example, by virtue of comprising an “autonomously replicating sequence” or ARS, such as the well-known AMA1 sequence.

DNA methylation: DNA methylation is a biochemical process that is important for normal development in higher organisms. It involves the addition of a methyl group to the 5 position of the cytosine pyrimidine ring or the number 6 nitrogen of the adenine purine ring (cytosine and adenine are two of the four bases of DNA). This modification can be inherited through cell division. Adenine or cytosine methylation is part of the restriction modification system of many bacteria, in which specific DNA sequences are methylated periodically throughout the genome. A methylase is the enzyme that recognizes a specific sequence and methylates one of the bases in or near that sequence. Foreign DNAs (which are not methylated in this manner) that are introduced into the cell are degraded by sequence-specific restriction enzymes and cleaved. Bacterial genomic DNA is not recognized by these restriction enzymes. The methylation of native DNA acts as a sort of primitive immune system, allowing the bacteria to protect themselves from infection by bacteriophage. E. coli DNA adenine methyltransferase (Dam) is an enzyme of ˜32 kDa. The target recognition sequence for E. coli Dam is GATC, as the methylation occurs at the N6 position of the adenine in this sequence (G meATC).

Methylation-specific nuclease: As described above, adenine or cytosine methylation is part of the restriction modification system of many bacteria, in which specific DNA sequences are methylated periodically throughout the genome. Type IIM restriction endonucleases on the other hand are able to recognize and cut methylated DNA. DpnI is a methylation-specific nuclease from Diplococcus pneumoniae G41 that recognizes the sequence methylated by the Dam methylase at the N6 position of the adenine in this sequence (G meATC).

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Variant: The term “variant” means a polypeptide having enzyme activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

### DETAILED DESCRIPTION OF THE INVENTION

In its first aspect, the present invention relates to methods of providing site-specifically mutated variant polypeptides, the method comprising the steps of:
a) providing a methylated template autosomal filamentous fungal replicating double-stranded circular DNA vector comprising a parent polynucleotide encoding a parent polypeptide;
b) providing a pair of end-to-end non-overlapping PCR primers directed to the parent polynucleotide, wherein at least one primer is mutagenic;
c) performing a PCR amplification of the template vector with the pair of PCR primers to generate full-length vector mutated PCR fragments;
d) removing the template vector with a suitable methylation-specific nuclease;
e) circularizing the mutated PCR fragments by self-ligation; and
f) transforming the circularized mutated PCR fragments directly into a filamentous fungal host cell to express the variant polypeptides,
   wherein either the PCR primers are phosphorylated prior to the PCR amplification, or the PCR fragments are phosphorylated before or during the self-ligation step to allow end-to-end ligation of the primers to circularize the mutated PCR fragments.

In a preferred embodiment of the first aspect, the template autosomal filamentous fungal replicating double-stranded circular DNA vector is a plasmid which comprises an AMA1 fungal replication initiation sequence.

In another preferred embodiment, the at least one mutagenic primer is fully complementary to the parent polynucleotide to which it is directed, except for one or more site-specific point mutation(s) designed to encode one or more amino acid insertion, substitution or deletion in the resulting PCR fragment(s) encoding the variant polypeptides; preferably the variants comprise a substitution, deletion, and/or insertion at one or more (e.g., several) positions. In an embodiment, the number of amino acid substitutions, deletions and/or insertions introduced into a variant is not more than 10, e.g., 1, 2, 3, 4, 5, 6, 7, 8 or 9. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for enzyme activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The parent polypeptide may be a naturally occuring polypeptide or a hybrid polypeptide in which a region of one polypeptide is fused at the N-terminus or the C-terminus of a region of another polypeptide.

The parent polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Preferably, the end-to-end non-overlapping PCR primers are at least 20 nucleotides in length, preferably at least 25, 30, 35, 40, 45, or most preferably at least 50 nucleotides in length. It is preferred that the PCR primers are phosphorylated prior to the PCR amplification to allow end-to-end ligation of the primers to circularize the mutated PCR fragments or, alternatively, that the PCR fragments are phosphorylated before or during the self-ligation step to allow end-to-end ligation of the primers to circularize the mutated PCR fragments.

In a preferred embodiment, the methylated template autosomal filamentous fungal replicating double-stranded circular DNA vector is methylated in vivo or in vitro by a methylase that recognizes GATC; preferably the methylase is the Dam methylase from E. coli; more preferably the methylation-specific nuclease used to remove the template vector recognizes Dam methylation; most preferably the methylation-specific nuclease is Dpn1.

Additional steps in the method of the first aspect are, of course, envisioned, such as, one or more step of screening, selecting, producing and/or isolating the variant polypeptide(s) of interest. Preferably, the methods of the first aspect comprise at least one additional step of screening or selecting the expressed variant polypeptides to identify one or more variants having one or more altered characteristic(s) of interest, such as, altered thermostability, altered specific activity, altered substrate specificity, altered solubility, altered storage stability, altered co-factor dependency. Preferably the alteration is a higher or lower characteristic compared to the parent polypeptide, e.g., a higher thermostability.

### Sources of Polypeptides

A polypeptide may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptide may be a bacterial polypeptide. For example, the polypeptide may be a Gram-positive bacterial polypeptide such as a Bacillus, Clostridium, Enterococcus, Geobacifius, Lactobacillus, Lactococcus, Oceanobacillus, Staphylococcus, Streptococcus, or Streptomyces polypeptide having [enzyme] activity, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma polypeptide.

In one aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide.

In another aspect, the polypeptide is a Streptococcus equisimilis, Streptococcus pyogenes, Streptococcus uberis, or Streptococcus equi subsp. Zooepidemicus polypeptide.

In another aspect, the polypeptide is a Streptomyces achromogenes, Streptomyces avermitilis, Streptomyces coelicolor, Streptomyces griseus, or Streptomyces lividans polypeptide.

The polypeptide may be a fungal polypeptide. For example, the polypeptide may be a yeast polypeptide such as a Candida, Kluyveromyces, Pichia, Saccharomyces, Schizosaccharomyces, or Yarrowia polypeptide; or a filamentous fungal polypeptide such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thermomyces, Thielavia, Tolypocladium, Trichoderma, Trichophaea, Verticillium, Volvariella, or Xylaria polypeptide.

In another aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide.

In another aspect, the polypeptide is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thermomyces lanuginosus, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride polypeptide.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

A polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dania (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis crylllA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory systems are those that cause expression of the gene to be turned on or off in response to a chemical or physical stimulus, including the presence of a regulatory compound. In filamentous fungi, the Aspergillus nigerglucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked with the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any filamentous fungal cell useful in the recombinant production of a polypeptide of the present invention. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus aculeatus, Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787.

### Methods of Production

The present invention also relates to methods of producing a variant polypeptide produced by the methods of the present invention.

The present invention also relates to methods of producing a polypeptide of the present invention, comprising (a) cultivating a recombinant host cell of the present invention under conditions conducive for production of the polypeptide; and (b) recovering the polypeptide.

The host cells are cultivated in a nutrient medium suitable for production of the polypeptide using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

The present invention also relates to methods of producing a protein, comprising (a) cultivating a recombinant host cell comprising such polynucleotide; and (b) recovering the protein.

The protein may be native or heterologous to a host cell. The term “protein” is not meant herein to refer to a specific length of the encoded product and, therefore, encompasses peptides, oligopeptides, and polypeptides. The term “protein” also encompasses two or more polypeptides combined to form the encoded product. The proteins also include hybrid polypeptides and fused polypeptides.

Preferably, the polypeptide is a hormone, enzyme, receptor or portion thereof, antibody or portion thereof, or reporter. For example, the polypeptide may be a hydrolase, isomerase, ligase, lyase, oxidoreductase, or a transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or a beta-xylosidase. The encoding gene may be obtained from any prokaryotic, eukaryotic, or other source.

The present invention is further described by the following examples that should not be construed as limiting the scope of the invention.

### Examples

### Example 1

### Site-Directed Variants Made Directly in Aspergillus

Enzyme variants with site-specific mutations have usually first been constructed at DNA-level in a standard intermediate host, such as E. coli. After the variants were made and verified in E. coli, the plasmid containing the encoding genes were then transformed into a filamentous fungal host, such as, an Aspergillus cell, where they were expressed.

The method outlined in the example below makes it possible to skip the early step in E. coli and move directly into a filamentous fungal host. The method is automatable and does not require, for example, restreaking of Aspergillus, because it is based on an autosomal replicating plasmid, which does not integrate into the Aspergillus chromosome.

### Mutation:

An N-glycosylation site was removed from a lipase enzyme by the introduction of a site-directed mutation (N33Q) using the claimed method. The absence of glycosylation in the resulting expressed variant polypeptide made it easy in this case to verify on an SDS-page gel that the substitution had taken place in the mutated variant.

### Phosphorylation of Oligo:

The following two oligos were phosphorylated using T4 polynucleotide kinase in T4 ligase buffer for 2 hours under condition recommended by manufacturer (New England biolab). 5 microliter of each of 50 microliter phosphorylation mixtures was used in a subsequent 100 microliter PCR reaction.

Primer 24885:
(SEQ ID NO: 1)
CCAGCTGGTACACAGATTACTTGCACGGGAAATGC
Primer 130411jvi8:
(SEQ ID NO: 2)
GGCATCATTGTTTTTTCCGCAG

### PCR Design:

A number of PCR's were run overnight using PHUSION™ polymerase and the templates mentioned in table 1 below.

PCR Program:

98° C. 20 seconds

25×(98° C. 20 seconds, 55° C. 20 seconds, 72° C. 5 minutes)

72° C. 7 minutes

### Template and Methylation:

Both of the template plasmids, pENI4286 and pENI1849 (FIG. 1), contain the AMA replication initiation region, thus ensuring that it can replicate in Aspergillus (see WO2003070956) as well as a lipase gene from Thermomyces lanuginosus. There are only minor sequence differences between the two plasmids

The plasmids have to be methylated at the A in the sequence GATC, in order to cut the template with DpnI at a later stage in the process. This can either be done in vivo or in vitro by a methylase such as Dam methylase, that recognizes GATC. The templates used in this example were methylated in vivo at GATC.

Any methylase could be used to methylate the DNA as long as there is a corresponding restriction enzyme to cut the recognition site in the methylated template, for example, the McrBC endonuclease.

TABLE 1
PCR components in each tube. Note that in tube 7 and 8
the oligoes have not been phosphorylated prior to PCR.

| tube | template | oligo | oligo | comments |
|---|---|---|---|---|
| 5 | pENI1849 | 130411jvi8 | 24885 | pre phosphorylated |
| 6 | pENI4286 | 130411jvi8 | 24885 | pre phosphorylated |
| 7 | pENI1849 | 130411jvi8 | 24885 | Phosphorylated |
| | | | | during ligation |
| 8 | pENI4286 | 130411jvi8 | 24885 | Phosphorylated |
| | | | | during ligation |

### PCR and Phosphorylation:

A PCR fragment was created where the 5″end and the 3″end just needed to be ligated in order to create the plasmid with the desired mutation. However, in order to be able to ligate the ends, they needed to be phosphorylated first. Alternatively, the ends of the PCR fragment could be phosphorylated after the PCR but before or during the ligation step.

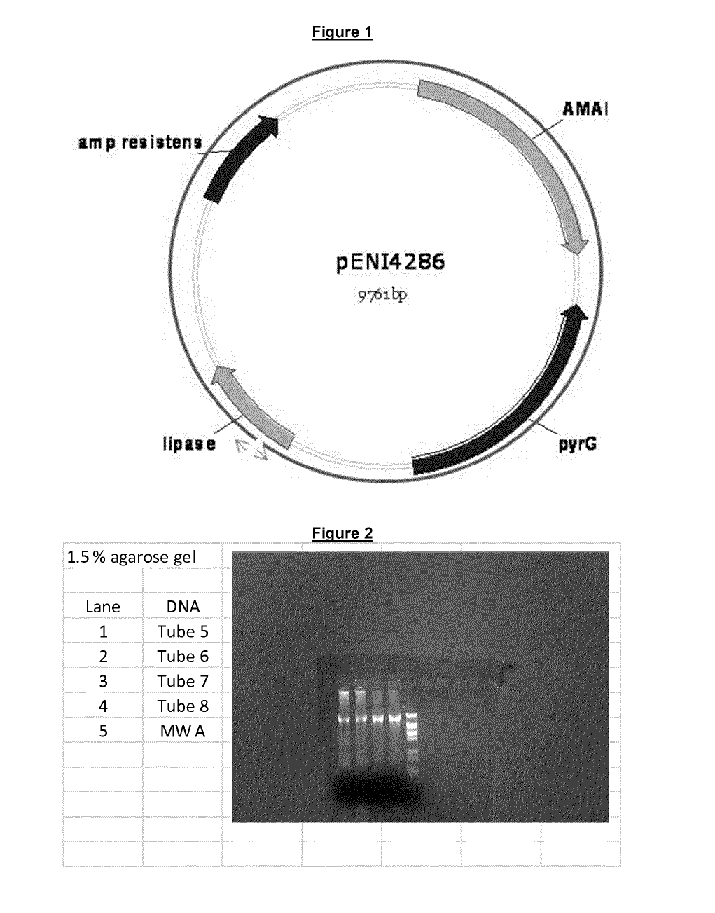

The PCR fragment size was verified on an agarose gel (FIG. 2).

### Removing Template:

25 microliter of a 5*NEB 4 buffer (New England Biolabs, USA) with DpnI was added to each PCR reaction and incubated for 3 hours at 37° C. in order to remove the original methylated templates.

### Purification of PCR Fragment:

50 microliter of the DpnI treated samples were purified on Biorad™ columns (Bio-Rad, USA) in order to exchange buffer. The DpnI step can be optimized in order to remove all the template, for example, by using less template, changing buffer prior to DpnI treatment and/or by prolonging incubation.

### Ligation and Phosphorylation:

10 microliter of a 5* T4 ligase buffer, T4 ligase and T4 polynucleotide kinase (all from New England Biolabs) was added to 40 microliter of biorad purified sample. The polynucleotide kinase was added to phosphorylate the PCR fragments generated in tube 7 and 8. This gave rise to phosphorylated PCR fragments, which were ligated by T4 Ligase; all in the same solution. The samples were set at 37° C. for 1 hour in order to phosphorylate the 5″ends and then the samples were moved to room temperature for 1 hour in order to ligate the ends.

5 microliter of ligation mixture was transformed into Aspergillus oryzae Toc1512 (as described in WO 98/01470 and WO2003070956) and plated. The plates were set at 37° C. over a weekend.

### Aspergillus Transformants:

4 transformants from each plate were inoculated in 200 microliter 2% YPM in a 96-well microtiterplate and incubated at 34° C. for 4 days without shaking.

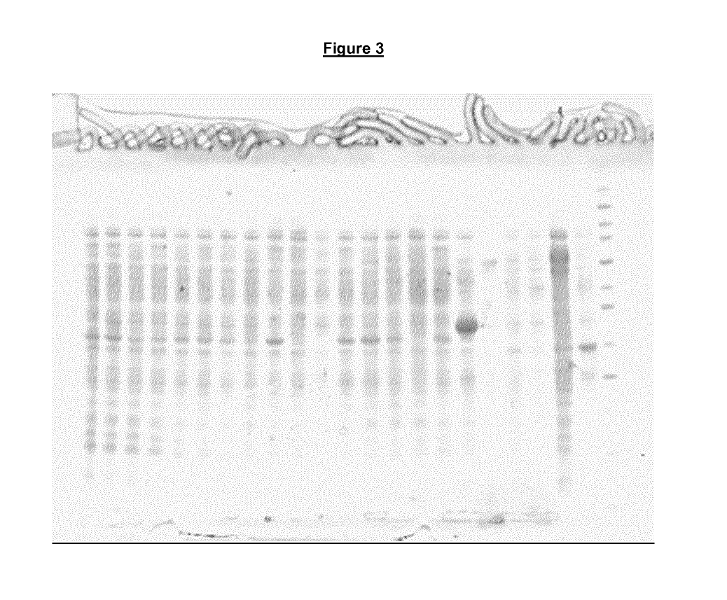

10 microliter of culture broth was loaded on an SDS page gel (10% Biorad gel, cat. No. 345-0113) as shown in FIG. 3 and a PNP-valerate assay on the Aspergillus samples was also made (as disclosed in WO 200024883). Comments to the SDS gel in FIG. 3 and the lipase activities measured are provided in table 2 below.

All variants seemed to be deglycosylated, indicating that the mutagenesis worked. The size of the SDS-PAGE bands correlated with the amount of activity. The method also worked when the oligos were phosphorylated during the ligation mixture (tubes 7 and 8).

TABLE 2
Overview of and comments to the SDS gel in FIG. 3 together
with lipase activities measured in the PNP-valerate assay.

| | | | Lipase activity | | |
|---|---|---|---|---|---|
| | | | (PNP-valerate | From | |
| Well | Lane | Clone | assay) | tube # | Comment |
| A1 | 1 | 1 | 122 | 5 | Not glycosylated |
| A2 | 2 | 2 | 177 | 5 | Not glycosylated |
| A3 | 3 | 3 | 37 | 5 | Not glycosylated |
| A4 | 4 | 4 | 104 | 5 | Not glycosylated |
| A5 | 5 | 5 | 141 | 6 | Not glycosylated |
| A6 | 6 | 6 | 156 | 6 | Not glycosylated |
| A7 | 7 | 7 | 142 | 6 | Not glycosylated |
| A8 | 8 | 8 | 104 | 6 | Not glycosylated |
| A9 | 9 | 9 | 288 | 7 | Not glycosylated |
| A10 | 10 | 10 | 70 | 7 | Not glycosylated |
| A11 | 11 | 11 | 56 | 7 | Not glycosylated |
| A12 | 12 | 12 | 358 | 7 | Not glycosylated |
| B1 | 13 | 13 | 145 | 8 | Not glycosylated |
| B2 | 14 | 14 | 92 | 8 | Not glycosylated |
| B3 | 15 | 15 | 35 | 8 | Not glycosylated |
| B4 | 16 | 16 | 126 | 8 | Not glycosylated |

| | | | |
|---|---|---|---|
| B5 | 17 | Lipase | Glycosylated control |

| | | |
|---|---|---|
| B6 | 18 | Not relevant |
| B7 | 19 | Not relevant |
| B8 | 20 | Not relevant |
| B9 | 21 | Not relevant |
| B10 | 22 | Not relevant |
| | 23 | Novex molecular weight marker |

1. A method of providing site-specifically mutated variant polypeptides, the method comprising the steps of:
a) providing a methylated template autosomal filamentous fungal replicating double-stranded circular DNA vector comprising a parent polynucleotide encoding a parent polypeptide;
b) providing a pair of end-to-end non-overlapping PCR primers directed to the parent polynucleotide, wherein at least one primer is mutagenic;
c) performing a PCR amplification of the template vector with the pair of PCR primers to generate full-length vector mutated PCR fragments;
d) removing the template vector with a suitable methylation-specific nuclease;
e) circularizing the mutated PCR fragments by self-ligation; and
f) transforming the circularized mutated PCR fragments directly into a filamentous fungal host cell to express the variant polypeptides,
wherein either the PCR primers are phosphorylated prior to the PCR amplification, or the PCR fragments are phosphorylated before or during the self-ligation step to allow end-to-end ligation of the primers to circularize the mutated PCR fragments.

2. The method of claim 1, wherein the parent polypeptide is an enzyme, preferably a hydrolase, isomerase, ligase, lyase, oxidoreductase, or a transferase; preferably the enzyme is an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or a beta-xylosidase.

3. The method of claim 1, wherein the template autosomal filamentous fungal replicating double-stranded circular DNA vector is a plasmid which comprises an AMA1 fungal replication initiation sequence.

4. The method of claim 1, wherein the at least one mutagenic primer is fully complementary to the parent polynucleotide to which it is directed, except for one or more site-specific point mutation(s) designed to encode one or more amino acid insertion, substitution or deletion in the resulting PCR fragment(s) encoding the variant polypeptides.

5. The method of claim 1, wherein each of the end-to-end non-overlapping PCR primers are at least 20 nucleotides in length, preferably at least 25, 30, 35, 40, 45, or most preferably at least 50 nucleotides in length.

6. The method of claim 1, wherein the methylated template autosomal filamentous fungal replicating double-stranded circular DNA vector is methylated in vivo or in vitro by a methylase that recognizes GATC; preferably the methylase is Dam.

7. The method of claim 6, wherein the methylation-specific nuclease used to remove the template vector recognizes Dam methylation; preferably the methylation-specific nuclease is Dpn1.

8. The method of claim 1, wherein the PCR primers are phosphorylated prior to the PCR amplification to allow end-to-end ligation of the primers to circularize the mutated PCR fragments.

9. The method of claim 1, wherein the PCR fragments are phosphorylated before or during the self-ligation step to allow end-to-end ligation of the primers to circularize the mutated PCR fragments.

10. The method of claim 1, wherein the filamentous fungal host cell is an Aspergillus cell; preferably the Aspergillus cell is an Aspergillus aculeatus, Aspergillus awamori, Aspergillus foetidus, Aspergillus fumigatus, Aspergillus japonicus, Aspergillus nidulans, Aspergillus niger or an Aspergillus oryzae cell.

11. The method of claim 1, which comprises at least one additional step of screening or selecting the expressed variant polypeptides to identify one or more variants having one or more altered characteristic(s) of interest.
